SEQUENCE LISTING (32 sequences)

SEQ ID NO: 1 - DNA, 1350 bp, Artificial sequence
  225 Inclonal heavy chain polynucleotide sequence
caggtccaactgcagcagccggggactgaactggtgaggcctggagcttcagtgaagctgtcctgcaaggcttctggcttctccttcaccagttactggatgaactgggtgaagcagaggcctggacaatgccttgagtggattggcatgattcatccttccgatagtgaaactaggttaaatcagaagttcaaggacagggccacattgactgtagacaaatcctccagcacagcctacatgcaactcagcagcccgacatctgaggactctgcggtctattactgtgcaagtgagatggattattactttgctatggactactggggtcaaggaacctcagtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggagaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 2 - protein, 449 aa, Artificial sequence
  T427 heavy chain polypeptide sequence
QVQLQQPGTELVRPGASVKLSCKASGFSFTSYWMNWVKQRPGQCLEWIGMIHPSDSETRLNQKFKDRATLTVDKSSSTAYMQLSSPTSEDSAVYYCASEMDYYFAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGRPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 657 bp, Artificial sequence
  T427 light chain polynucleotide sequence
gacattgtgctgacccaatctccaacttctttggctgtgtctctagggcagagggccaccatatcctgcagagccagtgaaagtgttgatagttatggcaatagttttatgcactggttccagcagaaaccaggacagccacccaaactcctcatctatcgtgcatccaacctagaatctgggatccctgccaggttcagtggcagtgggtcttggacagacttcaccctcaccattaatcctgtggaggctgatgatgttgcaacctattactgtcagcaaagtaatgaggatcctcggacgttcggtggaggcaccaaactggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 4 - protein, 218 aa, Artificial sequence
  T427 light chain polypeptide sequence
DIVLTQSPTSLAVSLGQRATISCRASESVDSYGNSFMHWFQQKPGQPPKLLIYRASNLESGIPARFSGSGSWTDFTLTINPVEADDVATYYCQQSNEDPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1350 bp, Artificial sequence
  225 Inclonal heavy chain polynucleotide sequence
caggtccaactgaagcagtctggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggaccctggtcaccgtttcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggagaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 6 - protein, 449 aa, Artificial sequence
  225 Inclonal heavy chain polypeptide sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGRPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 645 bp, Artificial sequence
  225 Inclonal light chain polynucleotide sequence
gacatcctactgacccagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattattgtcaacaaaataataactggccaaccacgttcggtgctgggaccaagctggagctcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 8 - protein, 214 aa, Artificial sequence
  225 Inclonal light chain polypeptide sequence
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 2406 bp, Artificial sequence
  T427 Inclonal heavy chain-PE38 fusion protein DNA coding sequence
atgcaggtccaactgcagcagccggggactgaactggtgaggcctggagcttcagtgaagctgtcctgcaaggcttctggcttctccttcaccagttactggatgaactgggtgaagcagaggcctggacaatgccttgagtggattggcatgattcatccttccgatagtgaaactaggttaaatcagaagttcaaggacagggccacattgactgtagacaaatcctccagcacagcctacatgcaactcagcagcccgacatctgaggactctgcggtctattactgtgcaagtgagatggattattactttgctatggactactggggtcaaggaacctcagtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggagaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaagcttccggaggtcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaa

SEQ ID NO: 10 - protein, 801 aa, Artificial sequence [sequence omitted]

SEQ ID NO: 11 - DNA, 1725 bp, Artificial sequence
  T427 Inclonal light chain-PE38 fusion protein DNA coding sequence
atggacattgtgctgacccaatctccaacttctttggctgtgtctctagggcagagggccaccatatcctgcagagccagtgaaagtgttgatagttatggcaatagttttatgcactggttccagcagaaaccaggacagccacccaaactcctcatctatcgtgcatccaacctagaatctgggatccctgccaggttcagtggcagtgggtcttggacagacttcaccctcaccattaatcctgtggaggctgatgatgttgcaacctattactgtcagcaaagtaatgaggatcctcggacgttcggtggaggcaccaaactggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggaaaagcttccggaggtcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaa

SEQ ID NO: 12 - protein, 574 aa, Artificial sequence
  T427 Inclonal light chain-PE38 fusion protein sequence
MDIVLTQSPTSLAVSLGQRATISCRASESVDSYGNSFMHWFQQKPGQPPKLLIYRASNLESGIPARFSGSGSWTDFTLTINPVEADDVATYYCQQSNEDPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 13 - DNA, 1104 bp, Artificial sequence
  CD30 extracellular domain coding sequence
atgaccttcccacaggatcgacccttcgaggacacctgtcatggaaaccccagccactactatgacaaggctgtcaggaggtgctgttaccgctgccccatggggctgttcccgacacagcagtgcccacagaggcctactgactgcaggaagcagtgtgagcctgactactacctggatgaggccgaccgctgtacagcctgcgtgacttgttctcgagacgacctcgtggagaagacgccgtgtgcatggaactcctcccgtgtctgcgaatgtcgacccggcatgttctgttccacgtctgccgtcaactcctgtgcccgctgcttcttccattctgtctgtccggcagggatgattgtcaagttcccaggcacggcgcagaagaacacggtctgtgagccggcttccccaggggtcagccctgcctgtgccagcccagagaactgcaaggaaccctccagtggcaccatcccccaggccaagcccaccccggtgtccccagcaacctccagtgccagcaccatgcctgtaagagggggcacccgcctcgcccaggaagctgcttctaaactgacgagggctcccgactctccctcctctgtgggaaggcctagttcagatccaggtctgtccccaacacagccatgcccagaggggtctggtgattgcagaaagcagtgtgagcccgactactacctggacgaggccggccgctgcacggcctgcgtgagctgttctcgagatgaccttgtggagaagacgccatgtgcatggaactcctcccgcacctgcgaatgtcgacctggcatgatctgtgccacatcagccaccaactcctgtgcccgctgtgtcccctacccaatctgtgcagcagagacggtcaccaagccccaggatatggctgagaaggacaccacctttgaggcgccacccctggggacccagccggactgcaaccccaccccagagaatggcgaggcgcctgccagcaccagccccactcagagcttgctggtggactcccaggccagtaagacgctgcccatcccaaccagcgctcccgtcgctctctcctccacggggaagcccgttctggattaa

SEQ ID NO: 14 - protein, 366 aa, Artificial sequence
  CD30 extracellular domain polypeptide sequence
MFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLD

SEQ ID NO: 15 - DNA, 32 bp, Artificial sequence
  Single strand DNA oligonucleotide
tttaaatcatgaccttcccacaggatcgaccc

SEQ ID NO: 16 - DNA, 35 bp, Artificial sequence
  Single strand DNA oligonucleotide
atatatgcggccgcttaatccagaacgggcttccc

SEQ ID NO: 17 - DNA, 30 bp, Artificial sequence
  Single strand DNA oligonucleotide
gatatacatatggaggtccaactgcagcag

SEQ ID NO: 18 - DNA, 34 bp, Artificial sequence
  Single strand DNA oligonucleotide
ccggatgcggccgcccgtttgatctccagcttgg

SEQ ID NO: 19 - DNA, 38 bp, Artificial sequence
  Single strand DNA oligonucleotide
ccacaggcgcgcactcccaggtccaactgcagcagccg

SEQ ID NO: 20 - DNA, 23 bp, Artificial sequence
  Single strand DNA oligonucleotide
ccactcaaggccttgtccaggcc

SEQ ID NO: 21 - DNA, 23 bp, Artificial sequence
  Single strand DNA oligonucleotide
ggacaaggccttgagtggattgg

SEQ ID NO: 22 - DNA, 31 bp, Artificial sequence
  Single strand DNA oligonucleotide
cttggtgctagctgaggagacggtgactgag

SEQ ID NO: 23 - DNA, 39 bp, Artificial sequence
  Single strand DNA oligonucleotide
ccacaggcgcgcactccgacattgtgctgacccaatctc

SEQ ID NO: 24 - DNA, 46 bp, Artificial sequence
  Single strand DNA oligonucleotide
agccaccgtacgtttgatttccagtttggtgcctccaccgaacgtc

SEQ ID NO: 25 - DNA, 20 bp, Artificial sequence
  Single strand DNA oligonucleotide
tgggcggtaggcgtgtacgg

SEQ ID NO: 26 - DNA, 33 bp, Artificial sequence
  Single strand DNA oligonucleotide
tttaaagaattccaacagatggctggcaactag

SEQ ID NO: 27 - DNA, 43 bp, Artificial sequence
  Single strand DNA oligonucleotide
tttaaagctagctgaggaaacggtgaccagggtcccttggccc

SEQ ID NO: 28 - DNA, 38 bp, Artificial sequence
  Single strand DNA oligonucleotide
tttaaacgtacgtttgagctccagcttggtcccagcac

SEQ ID NO: 29 - DNA, 27 bp, Artificial sequence
  Single strand DNA oligonucleotide
gacgtgagccacgaagaccctgaggtc

SEQ ID NO: 30 - DNA, 43 bp, Artificial sequence
  Single strand DNA oligonucleotide
aaatttgaattcacctccggaagctttacccggggacagggag

SEQ ID NO: 31 - DNA, 33 bp, Artificial sequence
  Single strand DNA oligonucleotide
aaacggcgtacggtggctgcaccatctgtcttc

SEQ ID NO: 32 - DNA, 55 bp, Artificial sequence
  Single strand DNA oligonucleotide
aaatttgaattcaccttcggaagcttttccaccgccacactctcccctgttgaag

(19) United States
(12) Patent Application Publication
(10) Pub. No.: US 2011/0003337 A1
(43) Pub. Date: Jan. 6, 2011

(54) IMMUNOGLOBULIN COMPOSITIONS AND METHODS OF PRODUCING SAME

(75) Inventors: Itai Benhar, Rechovot (IL); Rachel Hakim, Kibbutz Ramat Hakovesh (IL)

Correspondence Address: MARTIN D. MOYNIHAN d/b/a PRTSI, INC., P.O. BOX 16446, ARLINGTON, VA, 22215, US

(73) Assignee: Ramot at Tel-Aviv University Ltd., Tel-Aviv (IL)

(21) Appl. No.: 12/920,113

(22) PCT Filed: Feb. 25, 2009
(86) PCT No.: PCT/IL2009/000211

§ 371 Date: Aug. 30, 2010

Related U.S. Application Data
(60) Provisional application No. 61/064,372, filed on Feb. 29, 2008.

(51) Int. Cl. C12P 21/00; C07K 16/00

(52) U.S. Cl. 435/69.6; 530/387.3

(57) ABSTRACT

1. A method of producing an immunoglobulin in a bacterial culture, the method comprising:

(a) expressing in a first bacterial host cell a first polypeptide comprising an immunoglobulin light chain so as to form inclusion bodies which comprise the immunoglobulin light chain;

(b) expressing in a second bacterial host cell a second polypeptide comprising an immunoglobulin heavy chain so as to form inclusion bodies which comprise the immunoglobulin heavy chain;

(c) recovering from the inclusion bodies the first polypeptide and the second polypeptide so as to obtain reconstituted heavy chain and reconstituted light chain; and

(d) refolding the reconstituted heavy chain and reconstituted light chain under conditions which allow refolding of the reconstituted light chain and the reconstituted heavy chain predominantly as an intact immunoglobulin.

### FIELD AND BACKGROUND OF THE INVENTION

The present invention, in some embodiments thereof, relates to immunoglobulin compositions and methods of producing same.

Antibodies have recently become promising therapeutic proteins and are the leading categories of biopharmaceuticals with annual sales exceeding $ 20 billion. Successive technology waves have driven the growth of the monoclonal antibody sector, that was until recently dominated by chimeric antibodies such as Remicade and Rituxan, that will continue to drive market share through to 2008. However, the trend is shifting, with humanized and fully human monoclonal antibodies, together with recombinant antibody fragments such as Fabs, scFvs and conjugated antibodies, becoming increasingly important as drivers of the monoclonal antibody market growth at a forecast compound annual growth rate of 20.9%.

Although recombinant antibody fragments are making steady progress, the antibody market is still dominated by full-length IgG antibodies. While the small antibody fragments are usually produced in E. coli expression systems (Ward, 1993; Kipriyanov and Little, 1999), full-length monoclonal antibodies have traditionally been produced in mammalian cell culture due to their original hybridoma source and due to the complexity of the molecule (Simmons et al., 2002). A few alternative expression systems for full-length IgGs such as in plants (Ko and Koprowski, 2005) or in the milk of transgenic animals (Houdebine, 2002) do exist, but have not gained popularity (Kipriyanov and Le Gall, 2004).

Over 20 years ago, Cabilly and co-workers (Cabilly et al., 1984, Proc Natl Acad Sci USA 81, 3273-7) reported their attempt of producing IgG. They obtained a product of low quality and poor yield. This was one of the reasons that producing IgGs in E. coli was regarded impractical for more than two decades.

U.S. Pat. No. 6,331,415 discloses processes for producing an immunoglobulin or an immunologically functional immunoglobulin fragment containing at least the variable domains of the immunoglobulin heavy and light chains. The processes can use one or more vectors which produce both the heavy and light chains or fragments thereof in a single cell or in separate host cell cultures. The results provided (FIGS. 8B and 9) clearly show heterogenic products which are irrelevant for clinical applications.

Recently, the assembly of full-length antibodies in E. coli has been reported by two groups (Simmons et al., 2002; Mazor et al., 2007b). Both groups used vector systems directing the secretion of soluble heavy and light chains to the bacterial periplasm where they assembled into soluble active IgG molecules. The first study (Simmons et al., 2002, and U.S. Pat. No. 6,979,556, U.S. Patent Application 20070015244) produced antibody preparations that in addition to assembled IgG contained partially assembled species such as heavy chain dimers, or such dimers paired with only light chain. They concluded that the technology they described is a significant step in the process of efficiently producing full-length aglycosylated antibodies in E. coli, and that the yields are high enough to provide material for research purposes. They further suggested that the use of these proteins as therapeutic agents should be forthcoming with further increases in titer. The second study (Mazor et al., 2007b) improved upon the expression conditions using a somewhat different set of plasmids for secretion. They obtained IgGs of comparable quality, which still contained partially assembled species (Mazor et al 2007b, supplemental FIG. 4). The production yields reported by Mazor et al., 2007b were about 0.2-1 mg/l from low density shake flasks cultures, while Simmons et al., 2002 reported yields as high as 150 mg IgG/liters culture for high-density fermentations that after reaching a cell density of 40 OD₅₅₀ were induced for further 80 hours. It is difficult to make a comparison, but their yield is probably equivalent to a few mgs per shake flask culture.

U.S. 20080305516 teach methods for producing immunoglobulin molecules or immunologically functional immunoglobulin fragments which comprise at least functional portions of the variable domains of immunoglobulin heavy and light chains in Gram positive bacteria or eukaryotic cells. The methods comprise producing the heavy and the light chains in two separate host cells and refolding the immunoglobulin molecule or fragment ex vivo. The application clearly teaches away from expression of the antibodies in Gram negative cells, due to the difficulty in obtaining high molecular weight products in Gram negative cultures. Additionally, U.S. 20080305516 states that the result of producing large quantities of a desired protein in E. coli is often the formation of inclusion bodies and subsequent refolding which reduce the yields and quality.

Boss and co-workers (1984 Nucleic Acids Research 12:3791-3806) teach the production of murine IgM light and heavy chains in a single E. coli culture or separate E. coli cultures. Only residual antibody activity was detected upon reconstitution from inclusion bodies and refolding. The exhibited yield was poor as only radioactive-based analysis identified the product (see FIG. 3 therein).

### SUMMARY OF THE INVENTION

According to an aspect of some embodiments of the present invention there is provided a method of producing an immunoglobulin in a bacterial culture, the method comprising:

(a) expressing in a first bacterial host cell a first polypeptide comprising an immunoglobulin light chain so as to form inclusion bodies which comprise the immunoglobulin light chain;

(b) expressing in a second bacterial host cell a second polypeptide comprising an immunoglobulin heavy chain so as to form inclusion bodies which comprise the immunoglobulin heavy chain;

(c) recovering from the inclusion bodies the first polypeptide and the second polypeptide so as to obtain reconstituted heavy chain and reconstituted light chain; and

(d) refolding the reconstituted heavy chain and reconstituted light chain under conditions which allow refolding of the reconstituted light chain and the reconstituted heavy chain predominantly as an intact immunoglobulin.

According to some embodiments of the invention, the conditions comprise heavy-light chain molar ratio of about 1:2.

According to some embodiments of the invention, each of the first bacterial host and the second bacterial host are of Gram negative bacteria.

According to some embodiments of the invention, the Gram negative bacteria is E. coli.

According to some embodiments of the invention, the method further comprises purifying the immunoglobulin molecule on Protein A/G/L.

According to some embodiments of the invention, the method has a yield of at least 50 mg of purified immunoglobulin molecules per 1 liter of bacterial culture of the heavy chain, having an O.D.600 of 2.5 at the time of induction.

According to some embodiments of the invention, at least one of the first polypeptide and the second polypeptide comprises a therapeutic moiety.

According to some embodiments of the invention, at least one of the first polypeptide and the second polypeptide comprises an identifiable moiety.

According to some embodiments of the invention, the refolding predominantly as the intact immunoglobulin comprises at least 80% of the immunoglobulin as the intact immunoglobulin

According to some embodiments of the invention, the heavy chain is of the gamma family.

According to some embodiments of the invention, the light chain is of the kappa family.

According to some embodiments of the invention, the light chain is of the lambda family.

According to an aspect of some embodiments of the present invention there is provided an immunoglobulin produced according to the method described above.

According to an aspect of some embodiments of the present invention there is provided a composition-of-matter comprising a gram negative preparation remnants and at least 90% immunoglobulin.

According to some embodiments of the invention, the composition comprises no more than 10% immunoglobulin fragments.

According to some embodiments of the invention, the immunoglobulin is selected from the group consisting of IgA, IgD, IgE and IgG.

According to some embodiments of the invention, the IgG comprises IgG1, IgG2, IgG3 or IgG4.

According to some embodiments of the invention, the immunoglobulin is selected from the group consisting of a chimeric antibody, a humanized antibody and a fully human antibody.

According to some embodiments of the invention, the immunoglobulin is a bispecific antibody.

According to some embodiments of the invention, the immunoglobulin is selected from the group consisting of a primate immunoglobulin, a porcine immunoglobulin, a murine immunoglobulin, a bovine immunoglobulin, a goat immunoglobulin and an equine immunoglobulin.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of Inclonals expression vectors. Maps of plasmids pHAK-IgH for expression of human γ1 heavy chain; pHAK-IgL for expression of human κ light chain; pHAK-IgH-PE38 for expression of human γ1 heavy chain fused to a truncated form of Pseudomonas exotoxin A (PE38); pHAK-IgL-PE38 for expression of human κ light chain fused to PE38.

FIGS. 2A-B are protein gel images showing expression and purification of T427 Inclonal in E. coli. FIGS. 2a—12% SDS/PAGE. Lane 1, un-induced E. coli culture. Lane 2, induced heavy-chain. Lane 3, induced light-chain Lane 4, unpurified refolded IgG. Lane 5, Protein-A purified IgG. M, MW marker, in kDa, Lane 6, Cetuximab. Lane 7, protein-A purified T427 Inclonal. Lanes 1-5 were analyzed under reducing conditions while lanes 6-7 were not. Proteins were visualized by staining with GelCode Blue®. FIG. 2B is an immunoblot using HRP-conjugated anti human antibody and ECL development. The lane arrangement is as in A, except lane E=Cetuximab®.

FIG. 3 is a graph showing analysis of T427 Inclonal vs Cetuximab® by gel filtration chromatography. IgG samples were separated on a TSK3000 column. The arrows mark the migration pattern of commercial size markers on the column.

FIGS. 4A-C are graphs showing the binding properties of T427 Inclonal. FIG. 4a shows binding to MBP-CD30 as determined by an ELISA assay whereby detection is with HRP-conjugated anti human IgG. FIG. 4B shows binding of the T427 Inclonal antibody as determined by FACS analysis. Left panel—stable A431/CD30 transfected cells (left panel, 1) were incubated with 10 nM of chT427-IgG produced in mammalian cells or with T427 Inclonal. Right panel—FACS analysis of T427 Inclonal binding in the presence of ×30 molar excess of T427(dsFv)-PE38 immunotoxin as competitor. Binding was detected using FITC-conjugated anti human antibody. FIG. 4C is a graph showing specific cytotoxicity of T427-ZZ-PE38. A431/CD30 cells were incubated for 48 h with the indicated concentration of IgG-ZZ-PE38 immunoconjugate or with the T427 IgGs alone. The relative number of viable cells was determined using an enzymatic MTT assay. Each point represents the mean of a set of data determined in triplicate in three independent experiments. The error bars represent standard deviations of the data.

FIGS. 5A-C schematically illustrate the IgG-toxin fusion proteins and their resolution by SDS-PAGE. FIG. 5A is a schematic representation of the inclonals that were produced in this study. FIG. 5B is an immunoblot of protein-A-purified T427 Inclonals under non-reducing conditions. Lane 1, IgG; Lane 2, IgG-(di)-PE38; Lane 3, IgG-(tetra)-PE38; FIG. 5C is an immunoblot of protein-A-purified T427 Inclonal-PE38 fusion proteins under reducing conditions. Lane 1, IgG-(di)-PE38; Lane 2, IgG-(tetra)-PE38. M, MW marker, in kDa.

FIGS. 6A-C are graphs characterizing the T427 Inclonal-toxin fusion proteins. FIG. 6A shows evaluation of binding to MBP-CD30 in ELISA. Detection is with mouse anti PE serum mixed with HRP-conjugated goat anti mouse IgG. FIG. 6B shows the specific cytotoxicity of the inclonal-toxin fusions: A431/CD30 cells were incubated for 48 h with the indicated concentration of recombinant IgG-PE38 fusion proteins or T427(dsFv)-PE38 as a reference. The relative number of viable cells was determined using an enzymatic MTT assay. Each point represents the mean of a set of data determined in triplicate in three independent experiments. The error bars represent standard deviations of the data.

FIGS. 7A-B are graphs showing the binding properties of the anti EGFR 225 Inclonal. FIG. 7A—Binding to EGFR expressed on A431 cells tested by whole cell ELISA. Detection is with HRP-conjugated anti human IgG. FIG. 7B—FACS analysis: (b1) A431 cells were incubated with 10 nM of 225 Inclonal or the commercial anti EGFR antibody Cetuximab® used as control. (b2) FACS analysis as in b1 on G43 melanoma cells that do not express EGFR. (b3) FACS analysis of 225 Inclonal binding to A431 cells in the presence of ×30 molar excess of 225(scFv)-PE38 immunotoxin as competitor. Binding was detected using FITC-conjugated anti human antibody.

FIGS. 8A-C are graphs showing analysis of 225 Inclonal-ZZ-PE38. FIG. 8A—FACS analysis of EGFR expression levels of the cell lines: A431 (thick light grey), HEK293 (thick dark grey) and control G43 (thin light prey). EGFR levels were detected by staining with Cetuximab© mixed with FITC-conjugated anti human antibody. FIG. 8B—Cell-killing assay of 225 Inclonal-ZZ-PE38 on A431 cells (expressing a high level of EGFR). FIG. 8C—Cell-killing assay of 225 Inclonal-ZZ-PE38 on HEK293 cells (expressing a low level of EGFR) in which cells were incubated for 48 h with the indicated concentration of IgG-ZZ-PE38 immunoconjugates or the IgGs alone. The relative number of viable cells was determined using an enzymatic MTT assay. Each point represents the mean of a set of data determined in triplicate in three independent experiments. The error bars represent standard deviations of the data.

FIGS. 9A-B are graphs showing analysis of the stability of mammalian-cells produced T427 and of the T427 Inclonal upon incubation in 100% bovine serum. IgGs were diluted to a final concentration of 30 μg/ml in 100% bovine serum and incubated at 37° C. for the indicated time periods. Residual binding activity to MBP-CD30 of each feraction was evaluated by ELISA as described in FIG. 4A.

### DESCRIPTION OF EMBODIMENTS OF THE INVENTION

The present invention, in some embodiments thereof, relates to immunoglobulin compositions and methods of producing same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Recombinant antibodies have become a central modality in diagnostics and therapy and a large number of monoclonal antibodies are at various stages of clinical trial. Maximizing recombinant antibodies production yield is therefore highly desirable since it leads to cost efficient production.

Whilst reducing the present invention to practice, the present inventors have uncovered conditions which maximize production yield and assembly of immunoglobulins from prokaryotic cell systems when expressed as inclusion bodies. Recombinant antibody preparations produced according to the present teachings are highly homogeneous, whereby the predominant species in the preparation is an intact immunoglobulin while the presence of antibody fragments is only residual. The process described herein is simple in nature, cost-effective, can be easily scaled-up and characterized by relatively short duration between transformation, protein purification and refolding, rendering it effective for the industrial production of antibodies in bacteria.

Thus, according to an aspect of the invention there is provided a method of producing an immunoglobulin in a bacterial culture, the method comprising:

(a) expressing in a first bacterial host cell a first polypeptide comprising an immunoglobulin light chain so as to form inclusion bodies which comprise the immunoglobulin light chain;

(b) expressing in a second bacterial host cell a second polypeptide comprising an immunoglobulin heavy chain so as to form inclusion bodies which comprise the immunoglobulin heavy chain;

(c) recovering from the inclusion bodies the first polypeptide and the second polypeptide so as to obtain reconstituted heavy chain and reconstituted light chain; and

(d) refolding the reconstituted heavy chain and reconstituted light chain under conditions which allow refolding of the reconstituted light chain and the reconstituted heavy chain predominantly (i.e., more than 50%, 60%, 70%, 80%, 90% or more of antibody species) as the immunoglobulin.

As used herein “antibody species” refers to immunoglobulin and fragments of same (e.g., Fab, heavy chain monomers, light chain monomers, heteromer heavy-light chain).

The term “intact immunoglobulin” or “intact antibody” as used herein interchangeably, refers to whole antibodies produced by methods which are well known in the art which typically require immunization (see e.g., Harlow and Lane, Antibodies: A Laboratory Manual, Cold Spring Harbor Laboratory, New York, 1988, incorporated herein by reference). The antibody is typically a monoclonal antibody.

A whole or intact antibody comprises at least two heavy (H) chains and two light (L) chains inter-connected by disulfide bonds. Each heavy chain is comprised or a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The VH and VL regions can be further subdivided into regions of hypervariability, termed complementarity determining regions (CDRs), intersepted with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and the light chains contain a binding domain that interacts with the antigen. The constant regions of the antibody may mediate the binding of the immunoglobulin to host tissues or factors, including various cells of the immune system (e.g., effector cells) and the first component (Clq) of the classical complement system. Depending on the type of constant domain in the heavy chains, antibodies are assigned to one of five major classes: IgA, IgD, IgE, IgG, and IgM, and embodiments of the present invention envisages any of these or the following antibody subtypes and classes. Several of these are further divided into subclasses or isotypes, such as IgG1, IgG2, IgG3, IgG4, IgA1, IgA2, IgAsec and the like. The light chain may be of the “kappa” or “lambda” class. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG and/or IgM, commonly used in physiological/clinical situations are exemplary classes of antibodies for employment in this invention.

Antibodies of some embodiments of the present invention may be from any mammalian origin including human, porcine, murine, bovine, goat, equine, canine, feline, ovine and the like. The antibody may be a heterologous antibody. As used herein a “heterologous antibody” is defined in relation to a transgenic host such as a plant expressing said antibody.

According to some embodiments of the invention, the antibody is an isolated intact antibody (i.e., substantially free of cellular material other antibodies having different antigenic specificities and/or other chemicals).

As used herein “recombinant antibody” refers to intact antibodies that are prepared, expressed, created or isolated by recombinant means, such as (a0 antibodies isolated from an animal (e.g., mouse) that is transgenic for immunoglobulin genes (e.g., human immunoglobulin genes) or hybridoma prepared therefrom; (b) antibodies isolated from a host cell transformed to express the antibody; (c) antibodies isolated from a recombinant antibody library; and (d) antibodies prepared, expressed, created or isolated by any other means that involve splicing of immunoglobulin gene sequences to other DNA sequences. In certain embodiments immunoglobulin of the present invention may have variable and constant regions derived from human germline immunoglobulin sequences. In other embodiments, such recombinant human antibodies can be subjected to in vitro mutagenesis and thus the amino acid sequences of the VH and VL regions of the recombinant antibodies comprise sequences that while derived from and related to human germline VH and VL sequences, may not naturally exist within the human antibody germline repertoire in vivo.

The following exemplary embodiments of immunoglobulins are encompassed by the scope of the invention.

A used herein “human antibody” refers to intact antibodies having variable regions in which both the framework and CDR regions are derived from human germline immunoglobulin sequences as described, for example, by Kabat et al. (see Kabat 1991, Sequences of proteins of immunological Interest, 5^{th} Ed. NIH Publication No. 91-3242). The constant region of the human antibody is also derived from human germline immunoglobulin sequences. The human antibodies may include amino residues not encoded by human germline immunoglobulin sequences (e.g., mutations introduced by random or site directed mutagenesis in vitro or somatic mutation in vivo). However, the term “human antibody”, as used herein, is not intended to include antibodies in which CDRsequences derived from the germline of another mammalian species, such as a mouse, have been grafted onto human framework sequences.

As used herein “chimeric immunoglobulin” refers to an intact immunoglobulin or antibody in which the variable regions derive from a first species and the constant regions are derived from a second species. Chimeric immunoglobulins can be constructed by genetic engineering from immunoglobulin gene segments belonging to different species.

As used herein “humanized immunoglobulin” refers to an intact antibody in which the minimum mouse part from a non-human (e.g., murine) antibody is transplanted onto a human antibody; generally humanized antibodies are 5-10% mouse and 90-95% human.

In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

As used herein “bispecific” or “bifunctional antibody: refers to an artificial hybrid antibody having two different heavy/light chain pairs and two different binding sites. Bispecific antibodies can be produced by a variety of methods including fusion of hybridomas. See e.g., Songsivilai and Lachmann (1990) Clin. Exp. Immunol. 79:315-321; Kostelny et al. (1992) J. Immunol. 148:1547-1553.

The VH and VL sequences for application in this invention can be obtained from antibodies produced by any one of a variety of techniques known in the art.

Methods of producing polyclonal and monoclonal antibodies are well known in the art (See for example, Harlow and Lane, Antibodies: A Laboratory Manual, Cold Spring Harbor Laboratory, New York, 1988, incorporated herein by reference).

Typically, antibodies are provided by immunization of a non-human animal, preferably a mouse, with an immunogen comprising a desired antigen or immunogen. Alternatively, antibodies may be provided by selection of combinatorial libraries of immunoglobulins, as disclosed for instance in Ward et al (Nature 341 (1989) 544). Thus any method of antibody production is envisaged according to the present teachings as long as an immunoglobulin antibody is finally expressed in the bacterial host.

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988)). In a preferred embodiment, the non-human animal is a mammal, such as a rodent (e.g., mouse, rat, etc.), bovine, porcine, horse, rabbit, goat, sheep, etc. As mentioned, the non-human mammal may be genetically modified or engineered to produce “human” antibodies, such as the Xenomouse™ (Abgenix) or HuMAb-Mouse™ (Medarex). Typically, the immunogen is suspended or dissolved in a buffer, optionally with an adjuvant, such as complete Freund's adjuvant. Methods for determining the amount of immunogen, types of buffers and amounts of adjuvant are well known to those of skill in the art and are not limiting in any way on the present invention. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with adjuvant such as incomplete Freund's adjuvant. The recall injections, are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be utilized as long as they result in the production of B cells expressing an antibody directed to the antigen used in immunization.

In an alternate embodiment, lymphocytes from a non-immunized non-human mammal are isolated, grown in vitro, and then exposed to the immunogen in cell culture. The lymphocytes are then harvested and the fusion step described below is carried out.

For monoclonal antibodies, the next step is the isolation of splenocytes from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule and through a nylon mesh of a cell strainer into an appropriate buffer so as to produce a single cell suspension. The cells are washed, centrifuged and re-suspended in a buffer that lyses any red blood cells. The solution is again centrifuged and remaining lymphocytes in the pellet are finally re-suspended in fresh buffer.

Once isolated and present in single cell suspension, the lymphocytes are fused to an immortal cell line. This is typically a mouse myeloma cell line, although many other immortal cell lines useful for creating hybridomas are known in the art. Preferred murine myeloma lines include, but are not limited to, those derived from MOPC-21 and MPC-11 mouse tumors available from the Salk Institute Cell Distribution Center, San Diego, Calif. U.S.A., X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

The hybridomas are typically grown on a feeder layer of macrophages. The macrophages are preferably from littermates of the non-human mammal used to isolate splenocytes and are typically primed with incomplete Freund's adjuvant or the like several days before plating the hybridomas. Fusion methods are described in (Goding, “Monoclonal Antibodies: Principles and Practice,” pp. 59-103 (Academic Press, 1986

The cells are allowed to grow in the selection media for sufficient time for colony formation and antibody production. This is usually between 7 and 14 days. The hybridoma colonies are then assayed for the production of antibodies that bind the immunogen/antigen. The assay is typically a colorimetric ELISA-type assay, although any assay may be employed that can be adapted to the wells that the hybridomas are grown in. Other assays include immunoprecipitation and radioimmunoassay. The wells positive for the desired antibody production are examined to determine if one or more distinct colonies are present. If more than one colony is present, the cells may be recloned and grown to ensure that only a single cell has given rise to the colony producing the desired antibody. Positive wells with a single apparent colony are typically recloned and re-assayed to insure only one monoclonal antibody is being detected and produced.

Hybridomas that are confirmed to be producing a monoclonal antibody are then grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Amersham Biosciences, publication No. 18-1037-46, Edition AC, the disclosure of which is hereby incorporated by reference). The bound antibody is typically eluted from protein A, protein G or protein L columns by using low pH buffers (glycine or acetate buffers of pH 3.0 or less) with immediate neutralization of antibody-containing fractions. These fractions are pooled, dialyzed, and concentrated as needed.

DNA encoding the CDRs of the antibody of is readily isolated and sequenced using conventional procedures (e.g., by using oligonucleotide probes that are capable of binding specifically to genes encoding the heavy and light chains of antibodies such as murine or human). Once isolated, the DNA can be ligated into expression vectors, which are then transfected into bacterial host cells.

The DNA sequences encoding the immunoglobulin light chain and heavy chain polypeptides are independently inserted into separate recombinant vectors which may be any vector, which may conveniently be subjected to recombinant DNA procedures, and the choice of vector will often depend on the host cell into which it is to be introduced.

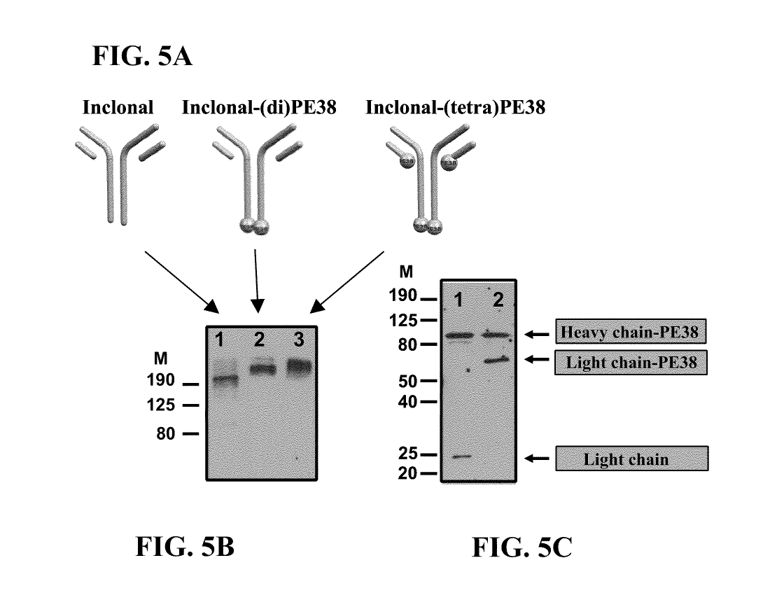

According to a specific embodiment the at least one of the heavy and light chain coding sequence further includes an in-frame sequence of a therapeutic or identifiable moiety, as to generate immunotoxins (e.g., used in therapeutic applications, such as in killing cancer cells) and immunolabels (e.g., used in diagnostic applications). Thus according to an embodiment of the invention the heavy chain comprises an in-frame fusion of the moiety, the light chain comprises an in-frame fusion of the moiety or both heavy and light chain comprise in-frame fusions of the moiety (see FIG. 5a).

The identifiable moiety can be a member of a binding pair, which is identifiable via its interaction with an additional member of the binding pair and a label which is directly visualized. In one example, the member of the binding pair is an antigen which is identified by a corresponding labeled antibody. In one example, the label is a fluorescent protein or an enzyme producing a colorimetric reaction.

The following Table 1 provides examples of sequences of identifiable moieties.

TABLE 1

| | Amino | |
|---|---|---|
| | Acid sequence | |
| | (Genebank | Nucleic Acid sequence |
| Identifiable Moiety | Accession No.) | (Genebank Accession No.) |
| Green Fluorescent protein | AAL33912 | AF435427 |
| Alkaline phosphatase | AAK73766 | AY042185 |
| Peroxidase | NP_568674 | NM_124071 |
| Histidine tag | AAK09208 | AF329457 |
| Myc tag | AF329457 | AF329457 |
| Biotin lygase tag | NP_561589 | NC_003366 |
| orange fluorescent protein | AAL33917 | AF435432 |
| Beta galactosidase | NM_125776 | NM_125776 |
| Fluorescein isothiocyanate | AAF22695 | AF098239 |
| Streptavidin | S11540 | S11540 |

The therapeutic moiety can be, for example, a cytotoxic moiety, a toxic moiety, a cytokine moiety and a bi-specific antibody moiety, examples of which are provided infra.

The following Table 2 provides examples of sequences of therapeutic moieties.

TABLE 2

| | Amino | |
|---|---|---|
| | Acid sequence | |
| | (Genebank | Nucleic Acid sequence |
| Therapeutic Moiety | Accession No.) | (Genebank Accession No.) |
| Pseudomonas exotoxin | AAB25018 | S53109 |
| (PE38) | | |
| Diphtheria toxin | E00489 | E00489 |
| interleukin 2 | CAA00227 | A02159 |
| CD3 | P07766 | X03884 |
| CD16 | AAK54251 | AF372455 |
| interleukin 4 | P20096 | ICRT4 |
| HLA-A2 | P01892 | K02883 |
| interleukin 10 | P22301 | M57627 |
| Ricin A toxin | 225988 | A23903 |

It will be appreciated that such fusions can also be effected using chemical conjugation (i.e., not by recombinant DNA technology).

The vector components generally include, but are not limited to, one or more of the following: a promoter, an origin of replication, one or more selection markers, and a transcription terminator sequence. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the immunoglobulin polypeptides is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

The bacterial host is selected capable of producing the recombinant proteins (i.e., heavy and light chains) as inclusion bodies (i.e., nuclear or cytoplasmic aggregates of stainable substances).

The host cells (e.g., first host cell and second host cell) used can be of identical species or different species.

According to specific embodiments of the present invention the host cells are selected from a Gran-negative bacterium/bacteria.

As used herein “Gram negative bacteria” refers to bacteria having characteristic staining properties under the microscope, where they either do not stain or are decolorized by alcohol during Gram's method of staining. Gram negative bacteria generally have the following characteristics: (i) their cell wall comprises only a few layers of peptidoglycans (which is present in much higher levels in Gram positive bacteria); (ii) the cells are surrounded by an outer membrane containing lipopolysaccharide (which consists of Lipid A, core polysaccharide, and O-polysaccharide) outside the peptideglycan layer; (iii) porins exist in the outer membrane, which act like pores for particular molecules; (iv) there is a space between the layers of peptidoglycan and the secondary cell membrane called the perimplasmic space; (v) the S-layer is directly attached to the outer membrane, rather than the peptidoglycan (vi) lipoproteins are attached to the polysaccharide backbone, whereas in Gram positive bacteria no lipoproteins are present.

Examples of Gram-negative bacteria which can be used in accordance with the present teachings include, but are not limited to, Escherichia coli Pseudomonas, erwinia and Serratia. It should be noted that the use of such Gram-negative bacteria other than E. coli such as pseudomonas as a host cell would provide great economic value owing to both the metabolic and physiologic properties of pseudomonas. Under certain conditions, pseudomonas, for example, can be grown to higher cell culture densities than E. coli thus providing potentially greater product yields.

The procedures used to ligate the DNA sequences coding for the polypeptides, the promoter (e.g., constitutive or inducible) and optionally the terminator sequence, respectively, and to insert them into suitable vectors containing the information necessary for replication, are well known to persons skilled in the art (see, for instance, Sambrook et al., Molecular Cloning: A Laboratory Manual, Cold Spring Harbor, N.Y., 1989).

Examples of bacterial expression vectors suitable for use in accordance with the present teachings include, but are not limited to, pET™ systems, the T7 systems and the pBAD™ system, which are well known in the art.

Methods of introducing expression vectors into bacterial host cells are well known in the art and mainly depend on the host system used.

The host cells can either be co-cultured in the same medium, or cultured separately.

Host cells are cultured under effective conditions, which allow for the expression of high amounts of recombinant heavy and light chain. Effective culture conditions include, but are not limited to, effective media, bioreactor, temperature, pH and oxygen conditions that permit recombinant protein production. An effective medium refers to any medium in which a bacterium is cultured to produce the recombinant protein of the present invention. Such a medium typically includes an aqueous solution having assimilable carbon, nitrogen and phosphate sources, and appropriate salts, minerals, metals and other nutrients, such as vitamins. Bacterial hosts of the present invention can be cultured in conventional fermentation bioreactors, shake flasks, test tubes, microtiter dishes, and petri plates, dependent on the desired amount. Culturing can be carried out at a temperature, pH and oxygen content appropriate for a recombinant host. Such culturing conditions are within the expertise of one of ordinary skill in the art.

Once appropriate expression levels of immunoglobulin heavy and light chains are obtained, the polypeptides are recovered from the inclusion bodies. Methods of recovering recombinant proteins from bacterial inclusion bodies are well known in the art and typically involve cell lysis followed by solubilization in denaturant [e.g., De Bernardez-Clark and Georgiou, “Inclusion bodies and recovery of proteins from the aggregated state” Protein Refolding Chapter 1:1-20 (1991). see also Examples section which follows,l under “Expression of Inclonals in E. coli”].

Briefly, the inclusion bodies can be separated from the bulk of cytoplasmic proteins by simple centrifugation giving an effective purification strategy. They can then be solubilized by strong denaturing agents like urea (e.g., 8 M) or guanidinium hydrochloride and sometimes with extremes of pH or temperature. The denaturat concentration, time and temperature of exposure should be standardized for each protein. Before complete solubilization, inclusion bodies can be washed with diluted solutions of denaturant and detergent to remove some of the contaminating proteins.

Finally, the solubilized inclusion bodies can be directly subjected to further purification through chromatographic techniques under denaturing conditions or the heavy and light chains may be refolded to native conformation before purification.

Thus, further purification of the reconstituted/refolded heavy and light chain polypeptides (i.e., solubilized reduced polypeptides) can be effected prior to and alternatively or additionally following refolding.

Methods of antibody purification are well known in the art and are described hereinabove and in the Examples section which follows. Other methods for purification of IgG are described in “Purification of IgG and insulin on supports grafted by sialic acid developing “thiophilic-like” interactions Hamid Lakhiaria and Daniel Mullerb, Journal of Chromatography B Volume 818, Issue 1, 15 Apr. 2005, Pages 53-59.

Alternatively or additionally, purification can be affinity-based through the identifiable or therapeutic moiety (e.g., using antibody columns which bind PE38).

To improve the refolding yield, the reconstituted heavy chain and reconstituted light chains are provided at a ratio selected to maximize the formation of immunoglobulin (i.e., intact). To this end, a heavy to light chain molar ratio of about 1:1 to 1:3, 1:1.5 to 1:3, 1:2 to 1:3 is. In an exemplary embodiment the heavy to light chain molar ratio is about 1:2.

Thus, embodiments of the above-described methodology produce immunoglobulins. Such immunoglobulins are referred to, also as inclonals. Such inclonals are provided in SEQ ID NOs. 1-12.

The above-described methodology is efficient for obtaining unprecedented yields of correctly folded, highly purified immunoglobulins from prokaryotic cells. Correct folding can be examined functionally and structurally. Methods of assaying activity are described at length in the Examples section which follows (e.g., antigen recognition, cell killing).

The present teachings provide immunoglobulin yield of at least 50 mg of purified immunoglobulin molecules per 1 liter of bacterial culture of the heavy chain, having an O.D.600 of 2.5 at the time of induction.

Thus, embodiments of the present invention provide for a composition-of-matter comprising a Gram negative preparation remnants (as described hereinabove such as comprising lipopolysaccharide) and at least about 70%, 80%, 85%, 90%, 95% or more immunoglobulin.

The composition of the invention preferably does not include more than 20%, 15%, 10%, 5% or even less antibody fragments (e.g., Fab, heavy chain monomers, light chain monomers, heteromer heavy-light chain, therapeutic moiety and identifiably moiety).

Gram negative remnants may be further removed for clinical applications (in vivo) using methods which are well known in the art/

When desired the immunoglobulin may be subjected to directed in vitro glycosylation, which can be done according to the method described by Isabelle Meynial-salles and Didier Combes. In vitro glycosylation of proteins: An enzymatic approach. Journal of Biotechnology Volume 46, Issue 1, 18 Apr. 1996, Pages 1-14.

Immunoglobulins and compositions (e.g., pharmaceutical composition) comprising same may be used in diagnostic and therapeutic applications and as such may be included in therapeutic or diagnostic kits.

Thus, compositions of the present invention may, if desired, be presented in a pack or dispenser device, such as an FDA approved kit, which may contain one or more unit dosage forms containing the active ingredient i.e., immunoglobulin. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Preparation of MBP-CD30 as a soluble antigen—Recombinant CD30 GENEBANK accession number AAA51947) was expressed as a maltose-binding protein fusion in E. coli. A DNA fragment corresponding to the extracellular domain of human CD30 (residues 51-383 of the full length gene product) was recovered by PCR using plasmid pHR30HNB [Rozemuller, H., Chowdhury, P. S., Pastan, I. & Kreitman, R. J. Isolation of new anti-CD30 scFvs from DNA-immunized mice by phage display and biologic activity of recombinant immunotoxins produced by fusion with truncated pseudomonas exotoxin. Int J Cancer 92, 861-870 (2001)] as template with primers CD30(N)-BspHI-FOR and CD30(N)-NotI-REV (All the PCR primers are described in Table 3, below).

TABLE 3

| Primer name | Sequences/SEQ ID NO: |
|---|---|
| CD30(N)-BspHI-FOR | 5′ TTTAAATCATGACCTTCCCACAGGATCGACCC/SEQ ID NO: 15 |
| CD30(N)-NotI-REV | 5′ ATATATGCGGCCGCTTAATCCAGAACGGGCTTCCC/SEQ ID NO: 16 |
| 225-NdeI-FOR | 5′ GATATACATATGGAGGTCCAACTGCAGCAG/SEQ ID NO: 17 |
| 225-NotI-REV | 5′ CCGGATGCGGCCGCCCGTTTGATCTCCAGCTTGG/SEQ ID NO: 18 |
| T427VH-BssHII-FOR | 5′ CCACAGGCGCGCACTCCCAGGTCCAACTGCAGCAGC CG/SEQ ID NO: 19 |
| T427VH-C44G-REV | 5′ CCACTCAAGGCCTTGTCCAGGCC/SEQ ID NO: 20 |
| T427VH-C44G-FOR | 5′ GGACAAGGCCTTGAGTGGATTGG/SEQ ID NO: 21 |
| T427VH-NheI-REV | 5′ CTTGGTGCTAGCTGAGGAGACGGTGACTGAG/SEQ ID NO: 22 |
| T427VL-BssHII-FOR | 5′ CCACAGGCGCGCACTCCGACATTGTGCTGACCCAATCTC/SEQ ID NO: 23 |
| T427VL-BsiWI-REV | 5′ AGCCACCGTACGTTTGATTTCCAGTTTGGTGCCTCCAC C GAACGTC/SEQ ID NO: 24 |
| CMV-Seq | 5′ TGGGCGGTAGGCGTGTACGG/SEQ ID NO: 25 |
| CMV-antiseq-EcoRI-REV | 5′ TTTAAAGAATTCCAACAGATGGCTGGCAACTAG/SEQ ID NO: 26 |
| 225VH-NheI-REV | 5′ TTTAAAGCTAGCTGAGGAAACGGTGACCAGGGTCCCT TGGCCC/SEQ ID NO: 27 |
| 225VK-BsiWII-REV | 5′ TTTAAACGTACGTTTGAGCTCCAGCTTGGTCCCAGCAC/SEQ ID NO: 28 |
| RGD/TAT-BsrGI-FOR | 5′ gacgtgagccacgaagaccctgaggtc/SEQ ID NO: 29 |
| CH3-HindIII-EcoRI-REV | 5′ AAATTTGAATTCACCTCCGGAAGCTTTACCCGGGGAC A GGGAG/SEQ ID NO: 30 |
| BsiWI-Back-IgL | 5′ AAACGGCGTACGGTGGCTGCACCATCTGTCTTC/SEQ ID NO: 31 |
| Cκ-HindIII-EcoRI-REV | 5′ AAATTTGAATTCACCTTCGGAAGCTTTTCCACCGCCA CACTCTC CCCTGTTGAAG/SEQ ID NO: 32 |

The PCR product was digested with BspHI and NotI and cloned into a pMALc-NHNN vector (pMALc-NHNN is a plasmid for the expression of MBP fusion proteins which was modified from pMALc-NN [originally described in Bach, H. et al. Escherichia coli maltose-binding protein as a molecular chaperone for recombinant intracellular cytoplasmic single-chain antibodies. J Mol Biol 312, 79-93 (2001)] by the addition of a HIS-tag on the 5′ end of the MBP coding sequence). The protein was produced and purified essentially as described for other MBP fusion proteins [Bach, H. et al. Escherichia coli maltose-binding protein as a molecular chaperone for recombinant intracellular cytoplasmic single-chain antibodies. J Mol Biol 312, 79-93 (2001)].

Preparation of the recombinant 225(scFv)-PE38 immunotoxin—The variable domains of anti EGFR mAb 225 were recovered by PCR using the plasmid pCMV/myc/ER-225(scFv) [Shaki-Loewenstein, S., Zfania, R., Hyland, S., Wels, W. S. & Benhar, I. A universal strategy for stable intracellular antibodies. J Immunol Methods 303, 19-39 (2005)] as template with the primers 225-NdeI-FOR and 225-NotI-REV. The PCR product was digested with NdeI and NotI and cloned into a derivative of the pRB98-Amp expression vector [Nagata, S. et al. Novel anti-CD30 recombinant immunotoxins containing disulfide-stabilized Fv fragments. Clin. Cancer Res. 8, 2345-2355 (2002)] that was linearized using the same enzymes. (In this derivative, the HindIII site used as 3′ end site for cloning scFvs was replaced with a NotI site to make it compatible for subcloning from common phage display vectors). The resulting plasmid, named pRB98-Amp-225(scFv)-PE38 was used for expression as a scFv-PE38 single-chain immunotoxin in BL21 (DE3) pUBS500 cells [Brinkmann, U., Mattes, R. E. & Buckel, P. High-level expression of recombinant genes in Escherichia coli is dependent on the availability of the dnaY gene product. Gene 85, 109-114 (1989)]. The expression, refolding and purification of the recombinant single-chain immunotoxin were preformed as described [Benhar, I. & Pastan, I. Cloning, expression and characterization of the Fv fragments of the anti-carbohydrate mAbs B1 and B5 as single-chain immunotoxins. Protein Eng 7, 1509-1515 (1994); Buchner, J., Pastan, I. & Brinkmann, U. A method for increasing the yield of properly folded recombinant fusion proteins: single-chain immunotoxins from renaturation of bacterial inclusion bodies. Anal Biochem 205, 263-270 (1992)]. The expression, refolding and purification of the recombinant immunotoxin T427(dsFv)-PE38 was preformed as described (Nagata, S., Onda, M., Numata, Y., Santora, K., Beers, R., Kreitman, R. J. and Pastan, I. (2002) Novel anti-CD30 recombinant immunotoxins containing disulfide-stabilized Fv fragments. Clin. Cancer Res. 8, 2345-55).

Construction of Vectors for Expression of Chimeric IgG1 in Mammalian Cells

Heavy chain vector: the VH variable domain of anti CD30 antibody T427 [Nagata, S. et al. Rapid grouping of monoclonal antibodies based on their topographical epitopes by a label-free competitive immunoassay. J Immunol Methods 292, 141-155 (2004)] was recovered by PCR using as template plasmid pRB98Amp-T427VH(C44)-PE38 (an expression vector for the VH-cys-PE38 component of the dsFv-immunotoxin). The 5′ end half of T427 VH was amplified using primers T427VH-BssHII-FOR and T427VH-C44G-REV that restore G44 (that was mutated to cys in the dsFv configuration). The 3′ end half of T427 VH was amplified using primers T427VH-C44G-FOR and T427VH-NheI-REV. The resulting PCR products were combined and assembled into an intact VH domain using primers T427VH-BssHII-FOR and T427VH-NheI-REV. The VH PCR product was digested with BssHII and NheI and cloned into a pMAZ-IgH vector [Mazor, Y., Barnea, I., Keydar, I. & Benhar, I. Antibody internalization studied using a novel IgG binding toxin fusion. J Immunol Methods 321, 41-59 (2007)] that was linearized using the same enzymes. The resulting plasmid, pMAZ-IgH-T427 was used to express the heavy chain of T427 (SEQ ID NOs: 1, 2) in a chimeric IgG1 format in mammalian cells.

Light chain vector: the V-Kappa variable domain of anti CD30 antibody T427 was recovered by PCR using as template plasmid pRB98Amp-T427VL(C105) (an expression vector for the VL-cys component of the dsFv-immunotoxin). The T427 VL was amplified using primers T427VL-BssHII-FOR and T427VL-BsiWI-REV that restored G105 (that was mutated to cys in the dsFv configuration). The VL PCR product was digested with BssHII and BsiWI and cloned into a pMAZ-IgL vector (Mazor et al. Supra) that was linearized using the same enzymes. The resulting plasmid, pMAZ-IgL-T427 was used to express the light chain of T427 (SEQ ID NOs: 3, 4) in a chimeric IgG1 format in mammalian cells.

Construction of Vectors for Expression of Inclonals

Heavy chain vectors: the VH variable domain of anti CD30 antibody T427 with the C-region of human IgG1 was subcloned from pMAZ-IgH-T427 (described above) into a T7-based, IPTG-inducible bacterial expression vector as follows: the entire heavy chain was amplified by PCR using plasmid pMAZ-IgH-T427 as template with primers CMV-Seq and CMV-antiseq-EcoRI-REV. The PCR product was digested with PstI and EcoRI and cloned into a pRB98Amp-T427VH(C44)-PE38 vector that was linearized using the same enzymes. The resulting plasmid, pHAK-IgH-T427 can be used to express the heavy chain of T427 in a chimeric IgG1 format in E. coli. VH domains can be exchanged into this plasmid as NdeI-NheI fragments. The DNA sequence of the T427 inclonal heavy chain is identical to the heavy chain sequence (supra) encoded by the mammalian expression vector pMAZ-IgH.

A similar plasmid for the expression of the heavy chain of the anti EGFR antibody 225 in E. coli was constructed as follows: the VH variable domain was recovered by PCR using plasmid pCMV/H6myc/cyto-225(Fv) [Shaki-Loewenstein, S., Zfania, R., Hyland, S., Wels, W. S. & Benhar, I. A universal strategy for stable intracellular antibodies. J Immunol Methods 303, 19-39 (2005)] as template with primers 225VH-NdeI-FOR and 225VH-NheI-REV. The PCR product was digested with NdeI and NheI and cloned into a pHAK-IgH-T427 vector (described above) that was linearized using the same enzymes. The resulting plasmid was named pHAK-IgH-225 (heavy chain SEQ ID NOs: 5, 6).

Light chain vectors: the light chain of anti CD30 antibody T427 with the human C-kappa region was subcloned from pMAZ-IgL-T427 (described above) into a T7-based, IPTG-inducible bacterial expression vector as follows: the entire light chain was amplified by PCR using plasmid pMAZ-IgL-T427 as template with primers CMV-Seq and CMV-antiseq-EcoRI-REV. The PCR product was digested with PstI and EcoRI and cloned into a pRB98Amp-T427VL(C105) plasmid vector that was linearized using the same enzymes. The resulting plasmid, pHAK-IgL-T427 can be used to express the light chain of T427 in a chimeric IgG1 format in E. coli. VL domains can be exchanged into this plasmid as NdeI-BsiWI fragments. The DNA sequence of the T427 inclonal light chain is identical to the light chain sequence (supra) encoded by the mammalian expression vector pMAZ-IgL.

A similar plasmid for the expression of the light chain of the anti EGFR antibody 225 in E. coli was constructed as follows: the V-Kappa variable domain was recovered by PCR using plasmid pCMV/H6myc/cyto-225(Fv) [Shaki-Loewenstein, S., Zfania, R., Hyland, S., Wels, W. S. & Benhar, I. A universal strategy for stable intracellular antibodies. J Immunol Methods 303, 19-39 (2005)] as template with primers 225VK-NdeI-FOR and 225VK-BsiWI-REV. the PCR product was digested with NdeI and BsiWI and cloned into a pHAK-IgL-T427 vector (described above) that was linearized using the same enzymes. The resulting plasmid was named pHAK-IgL-225 (SEQ ID NOs: 7 and 8).

Construction of vectors for the expression of IgG-PE38 fusion proteins—The heavy or light chain-PE38 fusion protein expression vectors were constructed on the backbone of pHAK vectors (supra) that were modified by insertion of HindIII and EcoRI cloning site at the 3′ end of the antibody constant regions as follows: For the heavy chain vector, the cloning site was inserted by PCR using plasmid pHAK-IgH as template with primers RGD/TAT-BsrGI-FOR and CH₃-HindIII-EcoRI-REV. For the heavy chain vector, pHAK-IgL was used as template with primers BsiWI-Back-IgL and Cκ-HindIII-EcoRI-REV. The PCR products were digested with BsrGI and EcoRI for the heavy chain and with SacI-EcoRI for the light chain, respectively, and cloned into a pHAK-IgH vector and pHAK-IgL vector respectively that were linearized using the same enzymes. The resulting vectors were linearized with HindIII and EcoRI and ligated with the PE38 DNA fragment that was recovered form plasmid pRB98Amp-T427VH(C44)-PE38 using the same enzymes. The resulting vectors were named pHAK-IgH-PE38 and pHAK-IgL-PE38.

Expression of chimeric IgGs in mammalian cells—The chimeric T427 IgG1 was expressed in HEK293 cells that were co-transfected with plasmids pMAZ-IgH-T427 and pMAZ-IgL-T427 and selected with G418 and hygromycin essentially as described (Mazor et al. Supra). After a highly-expressing clone was selected, it was expanded in DMEM supplemented with 10% FBS glutamine and antibiotics. 72 hours before harvesting, the cells were transferred into DCCM1 (serum free) medium (Beit-Haemek, Israel). Medium was collected several times at 48-72 h intervals. The IgG was purified from the conditioned medium by protein-A chromatography as described (Mazor et al. Supra). Protein concentrations of the purified proteins were determined by a Bradford assay (Coomassie Plus; Pierce, Rockford, Ill.) with BSA as the standard or by determination of absorbance at 280 nm and calculation of protein concentration based on its extinction coefficient. Purified IgG was stored at 4° C. Cetuximab® was purchased from Merck.

Expression of Inclonals in E. coli—The Inclonals and inclonal-PE38 fusion proteins were expressed in E. coli BL21(DE3) pUBS500 cells [Brinkmann, U., Mattes, R. E. & Buckel, P. High-level expression of recombinant genes in Escherichia coli is dependent on the availability of the dnaY gene product. Gene 85, 109-114 (1989)] that were transformed with the expression vectors. For the production of IgGs, cells were transformed with pHAK-IgH and pHAK-IgL. For the production of IgG-(di)PE38, cells were transformed with pHAK-IgH-PE38 and pHAK-IgL. For the production of IgG-(tetra)PE38, cells were transformed with pHAK-IgH-PE38 and pHAK-IgL-PE38. Cells were grown in SB medium (35 gr/L tryptone (Difco, USA), 20 gr/L yeast extract (Difco, USA), 5 gr/L NaCl, 6.3 gr/L glycerol (Frutarom, Israel), 12.5 gr/L K₂HPO₄, 3.8 gr/L KH₂PO₄, 0.48 gr/L MgSO₄, 0.4% (w/v) glucose) supplemented with 100 μg/ml ampicillin and 50 μg/ml kanamycin at 37° C. shaking at 250 RPM. The bacterial cultures were induced for protein expression in the late exponential growth phase (OD₆₀₀ of 2.5) with 1 mM isopropyl-1-thio-β-D-galactopyranoside for 3 h at 37° C. The recombinant proteins that accumulated as insoluble inclusion bodies, were isolated from lysed bacteria cells by centrifugation. From 500 ml of culture about 3 gr of wet cell paste was collected. The cells were suspended in 50 mM Tris (HCl) pH 8.0, 20 mM EDTA, using a tissuemizer. To lyse the cells, lysozyme was added to a final concentration of 500 mg/L and the cells were left at 25° C. for 1 h. The cell lysate was adjusted to 300 mM NaCl and 1.5% (v/v) triton X100 (SIGMA, Israel) and disrupted using a tissumizer. The insoluble fraction was collected by centrifugation at 10000 RPM, GSA rotor (Sorvall) for 30 min at 4 ° C. This crude inclusion bodies preparation was further purified by two additional cycles of homogenization in the same buffer (with 1% v/v TritonX100) followed by centrifugation. Finally, the inclusion bodies were treated once more in the same buffer with no detergent collected by centrifugation. The inclusion bodies were completely solubilized in 6 M guanidine hydrochloride, 50 mM Tris (HCl) pH 8.0, 20 mM EDTA and mixed at a heavy-light chain molar ratio of 1:2. From 1 liter of shake flask culture 200-300 mg of solubilized inclusion bodies protein were routinely obtained. The inclusion bodies mix was then reduced with 10 mg/ml dithioerythitol (SIGMA, Israel) (65 mM) for 2 h at 25° C.

Specifically, 50 mg of solubilized heavy chain protein (50 kDa MW) were mixed with 50 mg of solubilized light chain protein (25 kDa MW) before being reduced as a refolding mixture. In the case of PE38 fusion proteins, the relative ratios were adjusted to the molecular weight. For example, about 70 mg of solubilized heavy-chain-PE38 fusion protein (68 kDa MW) were mixed with about 30 mg of solubilized light chain (25 kDa MW).

The solubilized reduced proteins were refolded by 1:100 dilution into refolding solution containing redox shuffling and aggregation-preventing additives (0.1 M Tris (HCl) pH 9.5, 2 mM EDTA, 0.9 mM oxidized glutathione and 105 gr/L L-arginine) for 36 h at 8° C. After refolding, the protein was extensively dialyzed against phosphate/Urea buffer to a final pH of 7.4 (20 mM containing Na₂HPO₄ and NaH₂PO₄ and 100 mM Urea). The refolded active protein was then filter sterilized using a 0.45 μm filter and separated from contaminating bacterial proteins, excess light chains and from improperly folded protein by protein-A chromatography. Purified IgG was stored at 4° C. Typically, from a refolding initiated by mixing 50 mg of heavy chain with 50 mg of light chain protein, it is possible to obtain up to 15 mg of pure Inclonal.

The anti EGFR 225 Inclonal was produced in the same way using cultures of cells carrying pHAK-IgH-225 and pHAK-IgL-225.

Preparation of IgG-ZZ-PE38 immunocomplexes—The immunocomplex of chT427 or ch225 IgGs with ZZ-PE38 fusion protein was carried out by mixing IgGs with ZZ-PE38 fusion protein and purifying the immunocomplex by Superdex 200 (Amersham Pharmacia Biotech, now GE healthcare, USA) gel filtration chromatograph essentially as described (Mazor et al. Supra)

Gel filtration chromatography—Analytical separation of chimeric IgGs was carried out by gel-filtration chromatography using a 30 ml TSK3000 column (TosoHaas, Japan) on a fast protein liquid chromatography (FPLC), (Pharmacia LKB-Pump-P500) according to supplier's recommendations. About 200 micrograms of sample were loaded in 500 μl with PBS as buffer at a flow rate of 0.5 ml/min.

Evaluation of antigen binding by ELISA and whole-cell ELISA—Antigen binding by chimeric IgGs was tested in ELISA as follows: ELISA plates were coated with a solution of 5 μg/ml MBP-CD30 in PBS at 4° C. for 20 h and blocked with 3% (v/v) non-fat milk in PBS for 1-2 h at 37° C. All subsequent steps were done at room temperature (25° C.). Protein-A purified IgGs or IgG-PE38 fusion proteins were applied onto the plates in a five-fold dilution series and tested for their affinity to bind MBP-CD30. Following incubation the plates were washed three times with PBST. HRP-conjugated goat anti human antibodies (for IgGs) or mouse anti PE serum mixed with HRP-conjugated goat anti mouse antibodies (for IgG-PE38 fusion proteins) were used as secondary antibodies diluted ×5,000 dilution in PBST. The ELISA was developed using the chomogenic HRP substrate TMB and color development was terminated with 1 M H₂SO₄. The results were plotted as absorbance at 450 nm and the binding-avidity was roughly estimated as the IgG concentration that generates 50% of the maximal signal.

Cellular EGFR binding by 225 Inclonal and Cetuximab®, was tested by whole-cell ELISA as follows; the human epidermoid carcinoma A431 cells were seeded in 96-well plate at a density of 2×10⁴ cells/well in DMEM supplemented with 10% FBS for 16 h. The medium was aspirated and the cells were fixed with 3% glutaraldehyde for 15 minutes at 25° C. The wells were blocked with 3% (v/v) non-fat milk in PBS for 1-2 h at 37° C. Next, IgGs were added to the wells at a 5 fold dilution series in PBS+3% BSA and incubated for 1.5 h at 25° C. After cells were washed three time with PBS+3% BSA, 100 μl of HRP-conjugated goat anti human (×5000 dilution in PBS+3% BSA) was added for 1 h at 25° C. After another washing cycle, detection of cell bound antibodies was performed by addition of the chromogenic HRP substrate TMB to each well and color development was terminated with 1 M H₂SO₄. Absorbance was measured at 450 nm using a microplate reader.

Flow cytometry—Binding analysis of T427 based molecules to CD30 expressed on A431/CD30 transfected cells [Rozemuller, H., Chowdhury, P. S., Pastan, I. & Kreitman, R. J. Isolation of new anti-CD30 scFvs from DNA-immunized mice by phage display and biologic activity of recombinant immunotoxins produced by fusion with truncated pseudomonas exotoxin. Int J Cancer 92, 861-870 (2001)] with bacterial or mammalian produced chT427 IgG1 was tested by flow cytometry. Approximately 5×10⁵ cells in immunotubes (5 ml polystyrene tubes, Nunc, Sweden) were used in each experiment. After trypsinization, cells were washed once in 2% fetal calf serum in PBS (FACS buffer). Next, the chimeric IgGs were added at a final concentration of 10 nM in PBS+3% BSA and the cells were incubated for 1.5 h at 4° C. The cells were then washed three times FACS buffer and FITC-labeled goat anti human antibodies (×50 dilution in PBS+3% BSA) were added to the appropriate tubes for 45 min at 4° C. Detection of bound antibodies was done by flow cytometry on a FACS-Calibur (Becton Dickinson, Calif.) and results were analyzed with the CELLQuest program (Becton Dickinson). To confirm specificity, antibodies were incubated with or without a ×30 fold excess of competing protein during the 1.5 h incubation period.

Binding analysis of 225 based molecules to EGFR expressed on A431 cells was carried out in the same manner.

Cell-viability assay—The in vitro cell-killing activities of chimeric IgG-ZZ-PE38 immunocomplexes and of IgG-PE38 fusion proteins were measured by an MTT assay. Tested cells were seeded in 96-well plates at a density of 1×10⁴ cells/well in DMEM supplemented with 10% FBS. Immunocomplexes, IgG-PE38 fusion proteins or control proteins were added (in triplicate) in a 10 fold dilution series and the cells were incubated for 48 h at 37° C. in 5% CO₂ atmosphere. After 48 h, the media was replaced by fresh media (100 μl per well) containing 1 mg/ml MTT (Thiazolyl Blue Tetrazoliam Bromide, dissolved in PBS) reagent and the cells were incubated for another 4 h. MTT-formazan crystals were dissolved by the addition of 20% SDS, 50% DMF, pH 4.7 (100 μl per well) and incubation for 16 h at 37° C. Absorbance at 570 nm was recorded on an automated microtiter plate reader. The results are expressed as percentage of living cells relatively to the untreated controls that were processed simultaneously using the following equation: (OD₅₇₀ of treated sample/OD₅₇₀ of untreated sample)×100. The IC₅₀ values were defined as the immunocomplexes or the IgG-PE38 fusion protein concentrations that inhibited cell growth by 50%.

Evaluation of IgG Stability in Serum

To compare the stabilities of an Inclonal IgG T427 to that of the corresponding chT427 IgG that was produced in mammalian cell culture, a serum stability assay was carried out as follows: The IgGs were diluted to a final concentration of 30 μg/ml in 100% bovine serum (Beit Haemek, Israel) and incubated at 37° C. for the indicated time periods. Residual binding activity to MBP-CD30 of each fraction was evaluated by ELISA as described above.

Results

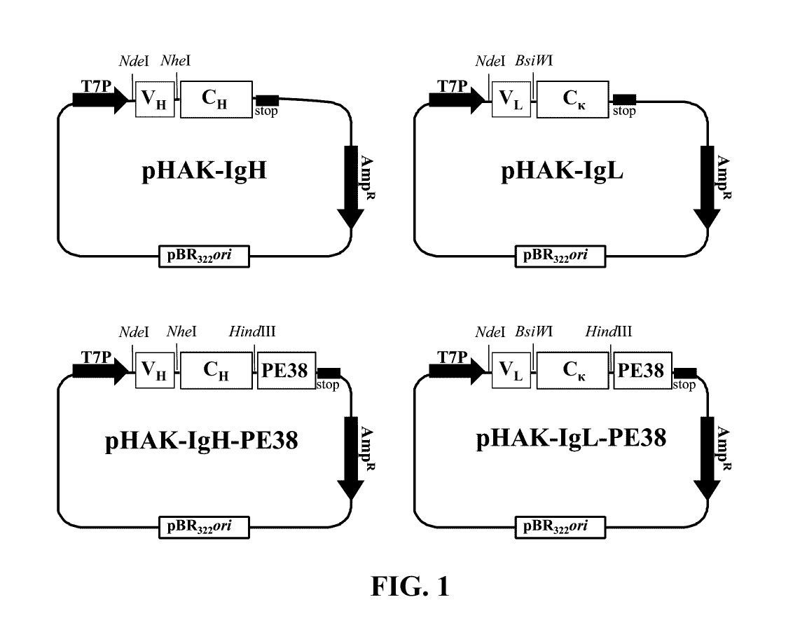

The present teachings provide for a highly efficient production method for full-length IgG and IgG-toxin fusion proteins in E. coli, named herein also “Inclonals”. This method involves expression of the proteins as insoluble inclusion bodies followed by refolding. A T7-based vector system was constructed for separate expression of the IgG heavy chain, light chain, or corresponding heavy or light chains that are fused to a truncated form of Pseudomonas exotoxin A (PE38). The expression vectors contained the constant regions of human gamma 1 heavy chain, and human kappa light chain, that were identical to those used for the construction of the mammalian-cell IgG expression system [Mazor, Y., Barnea, I., Keydar, I. & Benhar, I. Antibody internalization studied using a novel IgG binding toxin fusion. J Immunol Methods 321, 41-59 (2007), FIG. 1].

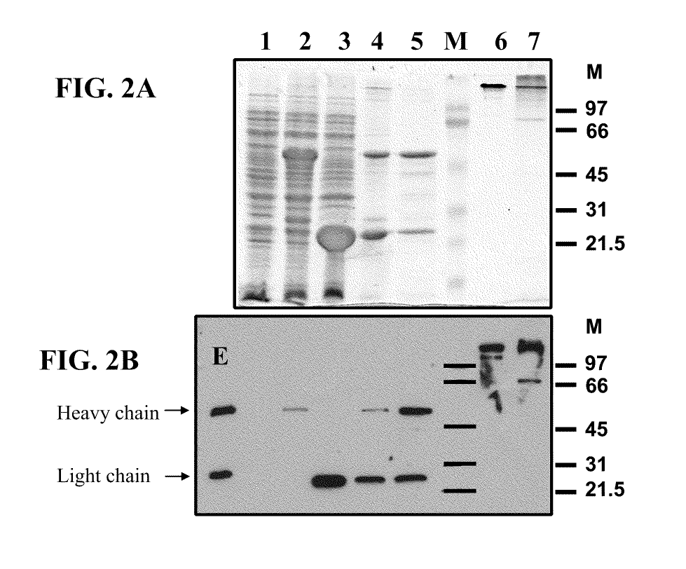

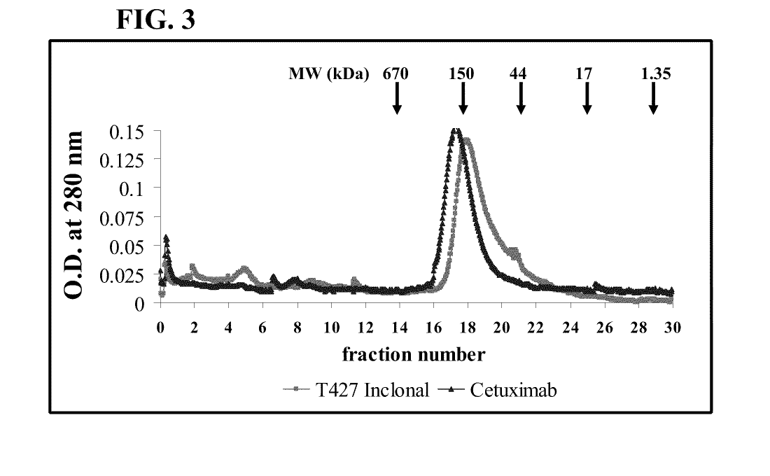

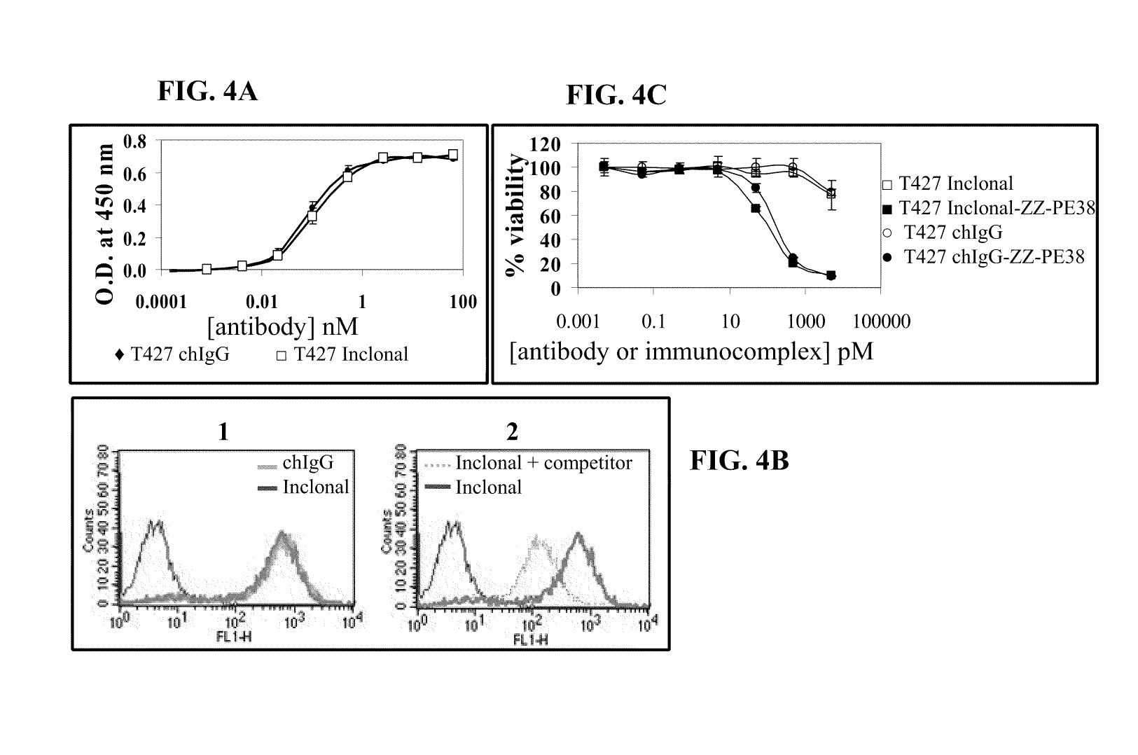

The model antibody was an anti CD30 antibody, T427 [Nagata, S. et al. Rapid grouping of monoclonal antibodies based on their topographical epitopes by a label-free competitive immunoassay. J Immunol Methods 292, 141-155 (2004)]. T427 was cloned into the expression vector and produced as described above. A high yield of highly purified preparation of chimeric T427 Inclonal was obtained (FIGS. 2a-b). For comparison, a batch of mammalian-cells produced of chimeric T427 IgG (T427 chIgG) was prepared essentially as described [Mazor et al. 2007, supra]. The bacterially produced Inclonal was compared to mammalian-cells produced IgG by gel-filtration chromatography, by antigen binding properties and by cell killing activity. An aliquot of the purified T427 Inclonal was analyzed by gel-filtration chromatography on a TSK3000 column. As shown (FIG. 3), the T427 Inclonal (calculated MW 147,500) eluted from the column as a monomer. The control mammalian-cell produced mAb Cetuximab® (calculated MW 151800) migrated as a slightly larger monomeric protein probably due to post-translational modifications (glycosylation) that are absent in the E. coli produced IgG. Antigen binding was studied by ELISA and by flow cytometry. As shown in FIG. 4a, the T427 Inclonal bound soluble antigen in ELISA with a similar avidity to the corresponding T427 chIgG that was produced in mammalian cell culture. Similarly, identical binding properties could be observed in the flow cytometry analysis on CD30-expressing cells (FIG. 4b1). Binding specificity could be demonstrated by the competition of the T427 Inclonal binding signal by a T427(dsFv)-PE38 recombinant immunotoxin (prepared as described above), as shown in (FIG. 4b2). The ability of the Inclonal antibodies to target tumor cells in vitro was evaluated by forming a complex with an antibody-binding toxin fusion protein (ZZ-PE38) (as described in Mazor et al. 2007, supra). The cytotoxicity evaluation also revealed that the T427 Inclonal parallels the performance of the mammalian cells produced antibody (FIG. 4c).

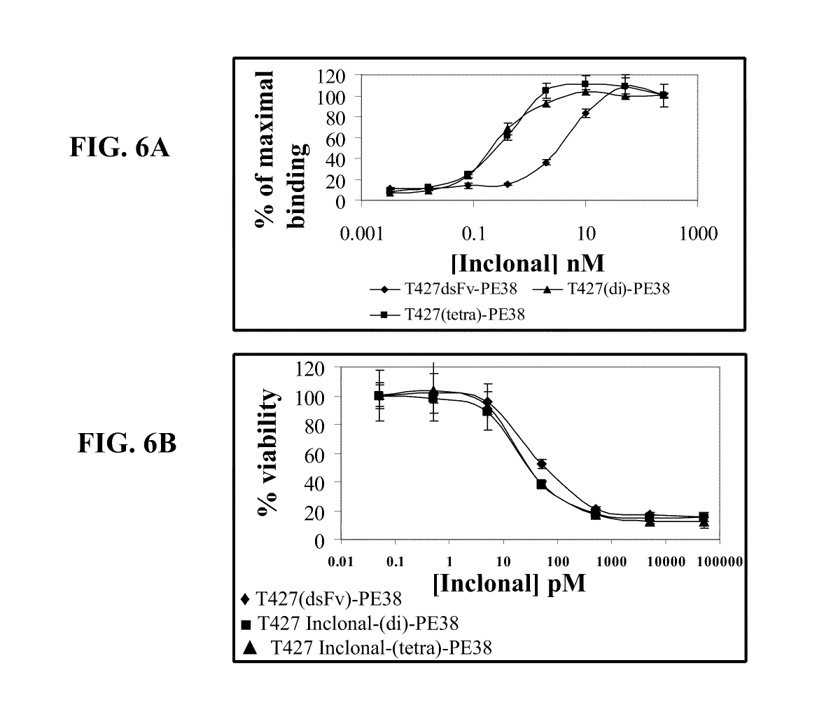

Thus, the present teachings provide the opportunity to generate full-length IgG that is genetically fused to a cytotoxic moiety, and consequently to explore IgG-enzyme fusion proteins. Pseudomonas exotoxin fusion proteins of the T427 Inclonal were prepared. Two derivatives were prepared; a T427(di)-PE38 derivative, with PE38 fused to the antibody heavy chain, and T427(tetra)-PE38, with PE38 fused to both the antibody heavy and light chains. The Inclonal-toxin fusion derivatives differ in their molecular weight (˜225 kDa for the di-toxin and ˜300 kDa for the tetra toxin) and in the number of toxin molecules payload delivered for each binding event (FIG. 5a). Both T427(di)-PE38 and T427(tetra)-PE38 were produced at a high purity (FIGS. 5b-c), and at a high yield, similar to that obtained for the IgG Inclonals. These novel proteins were evaluated for their binding properties and for their cell-killing activity. As shown (FIG. 6a), the apparent binding affinity as evaluated from the ELISA signal for both T427(di)-PE38 and T427(tetra)-PE38 is about 0.2 nM, which is similar to that of the T427 Inclonal and T427 chIgG (that are shown in FIG. 4a). Both IgGs bound with an apparent avidity, which was ×10 higher than the affinity of the corresponding monovalent recombinant immunotoxin T427(dsFv)-PE38.

The cell killing potential of T427(di)-PE38 and T427(tetra)-PE38 inclonal-toxin fusion proteins was tested on cultured CD30-expressing cells. As shown in FIG. 6b, both molecules inhibited the growth of the target cells with an IC₅₀ of ˜30 pM, while the monovalent immunotoxin T427(dsFv)-PE38 had an IC₅₀ of ˜60 pM.

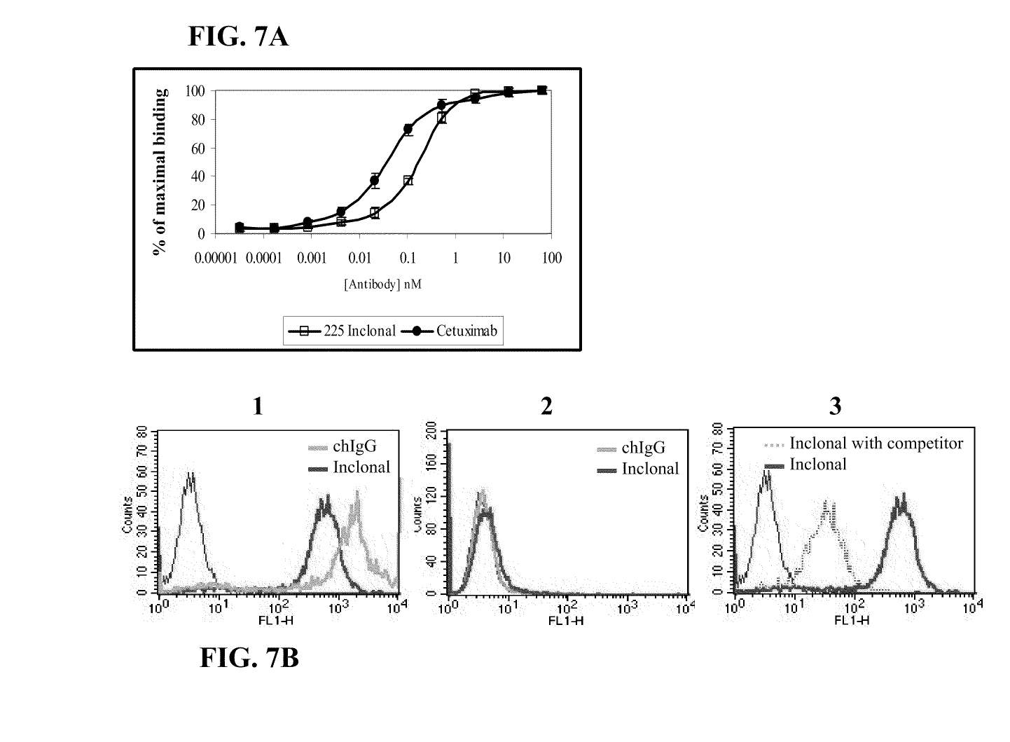

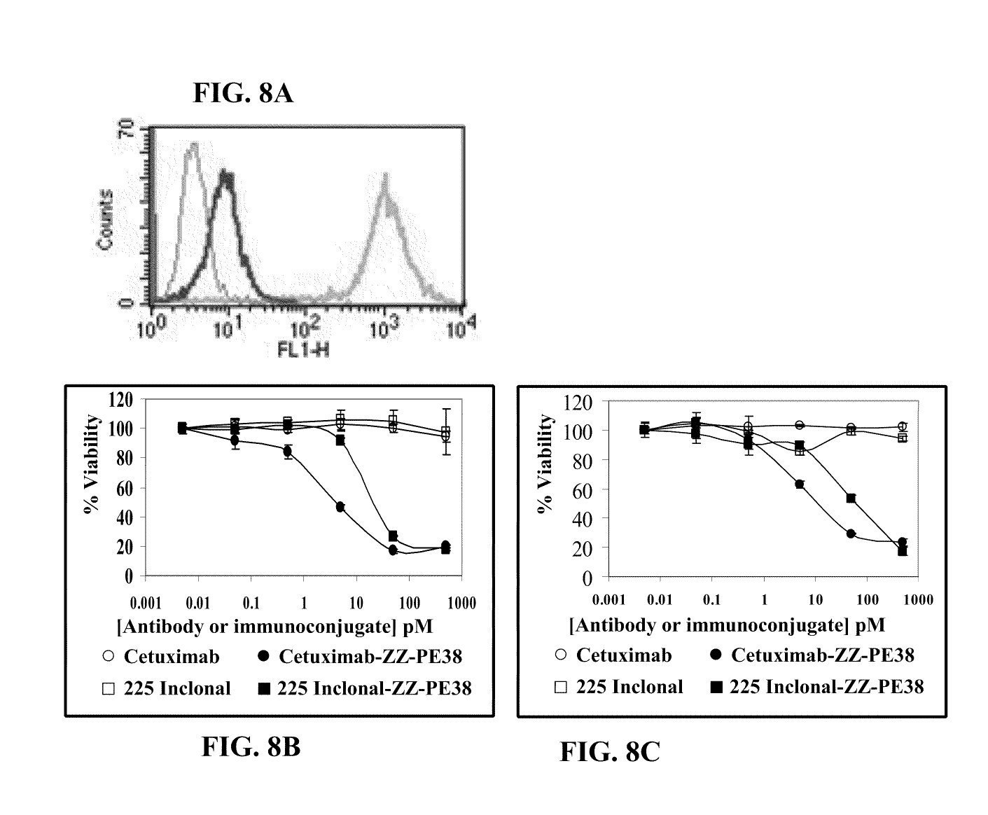

As an additional example, an Inclonal derivative of the anti EGF receptor antibody 225 was produced. MAb 225 is the parental mouse monoclonal antibody from which the therapeutic antibody Cetuximab® was derived [Rowinsky, E. K. The erbB family: targets for therapeutic development against cancer and therapeutic strategies using monoclonal antibodies and tyrosine kinase inhibitors. Annu Rev Med 55, 433-457 (2004)]. The 225 Inclonal was compared to Cetuximab® for antigen binding properties (FIGS. 7a-b) and by for cell killing activity as ZZ-PE38 immunocomplexes (FIGS. 8a-c). As shown in FIGS. 7a-b, the 225 Inclonal specifically bound EGFR expressing cells with about ×10 lower affinity than that of the Cetuximab®. Similarly, the 225 Inclonal-ZZ-PE38 immunocomplex had cytotoxic activity on both high EGFR expressing A431 cell line and on low EGFR expressing 293 cell line, which was about ×10 less potent than the Cetuximab®-ZZ-PE38 immunocomplex (FIGS. 8a-c). This difference is in accordance with the reported ×10 affinity increase reported for Cetuximab® in comparison to the 225 mAb [Rowinsky et al. supra].

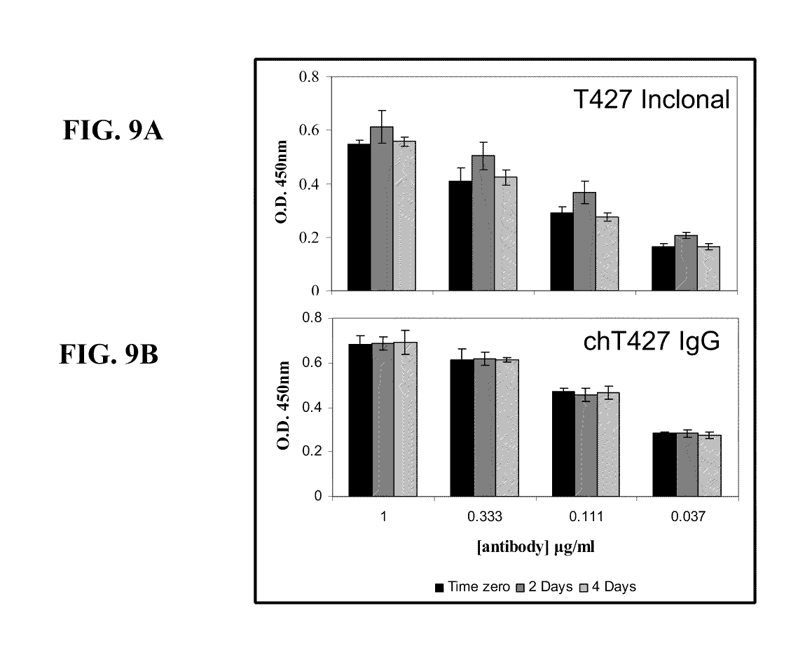

To further compare the quality of an Inclonal IgG T427 to that of the corresponding chT427 IgG that was produced in mammalian cell culture, a serum stability assay was carried out as follows: The IgGs were diluted to a final concentration of 30 μg/ml in 100% bovine serum (Beit Haemek, Israel) and incubated at 37° C. for the indicated time periods. Residual binding activity to MBP-CD30 of each fraction was evaluated by ELISA as described in FIG. 4a. As shown in FIG. 9a,b, the mammalian cell produced chT427 IgG and the T427 Inclonal were equally stable, losing no binding activity over the test period of 4 days at 37° C.

Embodiments of the invention demonstrate an expression and purification protocol developed for producing full-length IgGs and IgG-toxin fusion proteins, by refolding E. coli-produced inclusion bodies of the antibody heavy and light chain. By using this protocol, a yield of up to 50 mg pure IgG from 1 liter of shake flask culture and a highly purified product could be obtained. The Inclonals equaled the performance of the same IgGs that were produced using conventional mammalian cell culture in binding properties, as well as in their potential to deliver toxins to cultured target cells.

The Inclonals technology described herein, offers a rapid, generally applicable and potentially inexpensive method for the production of full-length antibodies. Most of the antibodies that can be potentially used for therapy, diagnostics or research purposes (such as virus neutralizing antibodies, antibodies that are used to ferry a cargo to the target cells, or bi-specific antibodies) are not dependent on Fc glycosylation to be effective. Moreover, embodiments of the present invention resolve the issue of conjugate heterogeneity and it should be applicable with a wide range of cytotoxic proteins. For research purposes, there is currently a great need to generate protein-specific affinity reagents to explore the human proteome. High-throughput methods to generate renewable antibodies are still immature [Uhlen, M., Graslund, S. & Sundstrom, M. A pilot project to generate affinity reagents to human proteins. Nat Methods 5, 854-855 (2008)]. Antibody-enzyme or antibody-fluorophore fusion proteins that can be generated by the “Inclonals” technology may become very useful for such purposes. It is believe that this rapid and cost effective IgG and IgG-fusion proteins production process and the high quality of the resultant product may make the bacterial production of full-length IgG and IgG-fusion proteins a viable and attractive option for antibody production.

Embodiments of the invention demonstrate that the modified expression-refolding system enables an effective production of full length IgGs in E. coli. By applying this novel system it was possible obtain two different antibodies; the anti-CD30 T427 antibody and the anti-EGFR 225 antibody (and more, data not shown). The production process of the antibody chains from inclusion bodies revealed high quantity of relatively pure protein. The entire refolding and purification process ended with up to 50 mg of IgG protein from 1 liter of shake flask culture, yields that were not reported before using bacterial expression systems for antibody production in low density culture. These production yields could benefit research laboratories that, in contrast to industrial laboratories, are generally not equipped with high density fermentors. Another important benefit of this system is the purity of the final product; following protein-A purification, the monomeric form of the antibody is notably the main form that was obtained. The purified protein is almost free of partially assembled species (that were observed in previous studies, Simmons 2002 and Mazor 2007, supra). The advantage of the E. coli production system in time consuming was considerable. The entire process in the mammalian system, from transfection, though selecting a highly-expressing clone, though expansion of the clone to IgG purification took about 8 weeks at best, while in the bacterial system, from transformation, though refolding to IgG purification it took only about 8-9 days.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of producing an immunoglobulin in a bacterial culture, the method comprising:
(a) expressing in a first bacterial host cell a first polypeptide comprising an immunoglobulin light chain so as to form inclusion bodies which comprise said immunoglobulin light chain;
(b) expressing in a second bacterial host cell a second polypeptide comprising an immunoglobulin heavy chain so as to form inclusion bodies which comprise said immunoglobulin heavy chain;
(c) recovering from said inclusion bodies said first polypeptide and said second polypeptide so as to obtain reconstituted heavy chain and reconstituted light chain; and
(d) refolding said reconstituted heavy chain and reconstituted light chain under conditions which allow refolding of said reconstituted light chain and said reconstituted heavy chain predominantly as an intact immunoglobulin, said conditions comprising arginine and wherein said conditions further comprise at least one of.

2. (canceled)

3. The method of claim 1, wherein each of said first bacterial host and said second bacterial host are of Gram negative bacteria.

4. The method of claim 3, wherein said Gram negative bacteria is E. coli.

5. The method of claim 1, further comprising purifying said immunoglobulin molecule on Protein A/G/L.

6. The method of claim 1, having a yield of at least 50 mg of purified immunoglobulin molecules per 1 liter of bacterial culture of the heavy chain, having an O.D.600 of 2.5 at the time of induction.

7. The method of claim 1, wherein at least one of said first polypeptide and said second polypeptide comprises a therapeutic moiety.

8. The method of claim 1, wherein at least one of said first polypeptide and said second polypeptide comprises an identifiable moiety.

9. The method of claim 1, wherein said refolding predominantly as said intact immunoglobulin comprises at least 80% of the immunoglobulin as said intact immunoglobulin.

10. The method of claim 1, wherein said heavy chain is of the gamma family and alternatively or additionally said light chain is of a family selected from kappa and lambda.

11-12. (canceled)

13. An immunoglobulin produced according to the method of claim 1.

14. A composition-of-matter comprising a gram negative preparation remnants and at least 90% immunoglobulin.

15. The composition-of-matter of claim 14, comprising no more than 10% immunoglobulin fragments.

16. The method of claim 1 wherein said immunoglobulin is selected from the group consisting of IgA, IgD, IgE and IgG.

17. The method of claim 16, wherein said IgG is selected from the group consisting of IgG1, IgG2, IgG3 or IgG4.

18. The method of claim 1, wherein said immunoglobulin is selected from the group consisting of a chimeric antibody, a humanized antibody and a fully human antibody.

19. The method of claim 1, wherein said immunoglobulin is a bispecific antibody.

20. The method of claim 1, wherein said immunoglobulin is selected from the group consisting of a primate immunoglobulin, a porcine immunoglobulin, a murine immunoglobulin, a bovine immunoglobulin, a goat immunoglobulin and an equine immunoglobulin.

21. The method of claim 1, wherein said conditions comprise an alkaline pH.

22. The method of claim 1, wherein said conditions comprise heavy-light chain molar ratio of about 1:2.

23. The method of claim 1, wherein said conditions comprise heavy-light chain molar ratio of about 1:2 and an alkaline pH.
